(19) United States
(12) Patent Application Publication
(10) Pub. No.: US 2016/0000464 A1
(43) Pub. Date: Jan. 7, 2016

(54) MEDICAL DEVICE FOR REDUCING BLEEDING

(71) Applicant: Arri COOMARASAMY, Birmingham (GB)

(72) Inventors: Arri Coomarasamy, Birmingham (GB); Richard Jameson, Royston (GB)

(21) Appl. No.: 14/768,379

(22) PCT Filed: Feb. 19, 2014
(86) PCT No.: PCT/GB2014/050479

§ 371 Date: Aug. 17, 2015

(30) Foreign Application Priority Data
Feb. 20, 2013 (GB) 1302977.2

(51) Int. Cl. A61B 17/42; A61B 19/04; A61B 17/12; A61B 19/02

(52) U.S. Cl. CPC A61B 17/42; A61B 19/026; A61B 19/04; A61B 17/1204; A61B 17/12099; A61B 17/12136; A61B 17/12159; A61B 2017/12004; A61B 2017/4216; A61B 2017/00557

(57) ABSTRACT

A medical device (20) is disclosed for reducing bleeding comprising an outer flexible membrane (21) and an inner flexible membrane (22), an inflatable region (25), an inflow channel (23) and an interior region (29). The inflatable region (25) is defined between the between the inner and outer flexible membrane (22, 21). The inflow channel (23) is for inflation of the inflatable region (25) by communicating a fluid thereto. The interior region (29) is within the inflatable region (25), and is defined by the inner flexible membrane (22). The interior region (29) is configured to receive an insertion means (9,22) for positioning the medical device (20). The medical device (20) may be used to reduce post-partum haemorrhage (PPH). A method of using the medical device to reduce PPH is disclosed.

The present invention relates to a medical device for reducing bleeding. More particularly, the present invention relates to a medical device for reducing bleeding from a womb.

Every year, approximately 14 million women (10% of all women who give birth) suffer with haemorrhage after child birth. One in a hundred of these die as a result of haemorrhage, and this accounts for about a third of all maternal deaths.

Uterine balloon tamponade devices are sometimes used to stop the bleeding by tamponading the inside of the womb. Examples of such devices are: Bakri Balloon, Sengstaken-Blakemore Tube, Rusch Balloon and Foley Catheter. All these devices share a common design of having a long tube attached to an inflatable balloon that can be placed inside the womb. The balloon is then inflated using a fluid introduced via the tube to provide pressure against the wall of the womb. Of these four devices, only the Bakri Balloon was specifically designed to stop post-partum haemorrhage.

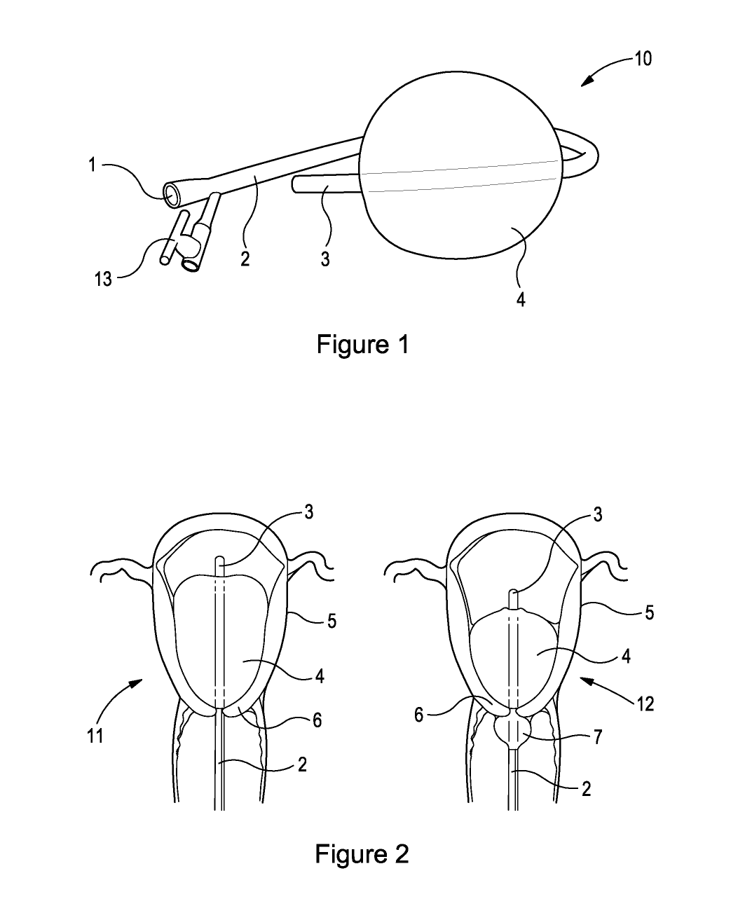

Referring to the appended drawings, FIG. 1 shows an example of a Bakri Balloon 10, comprising a substantially spherical inflated balloon 4, an outlet opening 1, inlet valve 13, combined inlet/outlet tube 2 and drainage portion 3. The balloon 4 can be inflated using a fluid introduced via the inlet valve 13, through the combined inlet/outlet tube, which carries two fluid conduits, a first conduit communicating between the interior of the balloon 4 and the inlet valve 13, and a second conduit communicating between a drainage port in the drainage portion 3. The drainage portion and second fluid conduit together form an outflow channel running through the balloon 4, which will reveal any bleeding. Blood collecting in the region of the drainage portion 3 will pass through the drainage port, out of the outflow channel where it will be become readily observable.

All of the prior art devices have several weaknesses. Firstly, it is difficult or impossible to achieve accurate placement of prior art balloon tamponade devices in the womb. The effectiveness of a uterine balloon is critically dependent on accurate placement of the device within the womb. It has hitherto been suggested by manufacturers of Bakri Balloons that ultrasound scanning may be used to achieve correct placement, but ultrasound scanning is not universally available during childbirth, and even when an ultrasound scanning machine is available, the appropriate technical expertise may not be. Furthermore, worldwide up to 70% of childbirth takes place outside a health facility, without access to ultrasound facilities.

A second problem with prior art devices is balloon slippage during inflation. During inflation of the balloon, prior art devices may slip down from the womb through the cervix, so that they are at least partially in the vagina, creating an “hour-glass” effect. This problem is illustrated in FIG. 2 of the appended drawings, in which a diagram 11 of a correctly placed balloon 4 according to FIG. 1 is shown, adjacent to a diagram 12 of a slipped, incorrectly placed balloon 4 according to FIG. 1. In both cases the balloon 4 is positioned within the uterus 5, with the combined inlet/outlet tube 4 passing through the cervix 6 and out of the body through the vagina. In the correctly positioned balloon shown in diagram 11, the balloon 4 is entirely within the uterus 5, and none of it protrudes into the vagina through the cervix 6. The correctly positioned balloon 4 exerts pressure on the appropriate regions of the uterus, and the drainage portion is positioned adjacent to the top of the uterus.

The incorrectly placed balloon 4 shown in diagram 12 has a portion 7 extending through the cervix 6 into the vagina. The balloon 4 subsequently takes on an “hour-glass” shape because of the constriction of the cervix 6 between the more open volumes of the uterus 5 and vagina respectively. The portion of the balloon 4 remaining in the uterus 5 is effectively under sized, and fails to exert the appropriate pressure on the appropriate regions of the uterus 5. Such slippage therefore makes the device less effective.

A third problem with prior art devices is the difficulty in judging the proper inflation pressure of the balloon. It is important to avoid both under- and over-inflation of the balloon. Over-inflation may cause damage to the womb or impair contraction of the womb. Under-inflation will not provide sufficient tamponade effect. An understanding of the balloon pressure within the womb would be useful to prevent both under- and over-inflation. None of the prior art devices allow assessment of intra-uterine pressure during inflation.

It is an object of the present invention to provide an improved medical device for reducing bleeding. It is a further object of the present invention to overcome, or at least ameliorate, some of the abovementioned problems.

According to the present invention, there is provided a medical device for reducing bleeding comprising: an outer flexible membrane and an inner flexible membrane, wherein the outer and inner flexible membranes together define an inflatable region between the inner and outer flexible membrane; an inflow channel for inflation of the inflatable region by communicating a fluid thereto; and an interior region defined by the inner flexible membrane, the interior region being configured to receive an insertion means for positioning the medical device.

The interior region may be at least partly within the inflatable region.

The medical device may further comprise a sleeve, communicating from a sleeve opening to the interior region.

The sleeve and/or interior region may be configured to receive at least part of a hand as the insertion means. The sleeve and/or interior region may be configured to receive at least one finger as the insertion means. Alternatively, the sleeve and/or interior region may be configured to receive an elongate insertion means that is more slender than a hand.

The sleeve may be configured to extend, in use, substantially as far as an operator's elbow

The medical device may further comprise an outflow channel configured to communicate fluid from an outflow region adjacent to the exterior of the inflatable region to a location remote from the outflow region. The outflow channel may communicate fluid from the outflow region to a location proximal to the sleeve opening.

The outflow channel may be disposed on the exterior of the inflatable region. The outflow channel may be disposed on the exterior of the sleeve.

The inflow channel may be at least partly defined by the inner flexible membrane and/or the outer flexible membrane.

The medical device may further comprise a reinforced region of the outer flexible membrane.

The reinforced region may define at least part of the inflatable region adjacent to the sleeve.

The sleeve may be at least partly defined by the inner flexible membrane and/or the outer flexible membrane.

The medical device may be configured for reducing postpartum haemorrhage.

The medical device, and in particular the inner and outer flexible membranes and the sleeve, if present, may be made of silicone, rubber, latex, polyurethane or any suitable material.

According to a second aspect of the invention, a medical apparatus is disclosed comprising a medical device according to a first aspect of the invention and: a sponge or plug configured to be inserted into the medical device to aid retention of the inflatable region, and/or an insertion means configured to enable insertion and/or positioning of the inflatable region within a region of the body into which a hand cannot be placed. The plug may be a separate inflatable plug.

The sponge may be a cervical sponge plug. Such vaginal packing may also provide a cervical and/or vaginal tamponade and help reduce bleeding from cervical and/or vaginal tears.

The insertion means may comprise a plastics and/or metal material. The insertion means may be configured to be held in the hand. The insertion means may be more slender than a hand, to enable insertion and/or positioning of the medical device and/or medical apparatus within more constricted body cavities.

According to a third aspect of the invention, there is provided a method of reducing bleeding using the device or apparatus according to a first or second aspect of the invention.

The method may comprise the steps of: inserting and/or positioning a medical device according to a first aspect of the invention within a body cavity, wherein inserting comprises using an insertion means received within the interior region of the medical device, and inflating the medical device.

The method may further comprise the steps of deflating the medical device and removing it from the body cavity.

The insertion means may be received within the interior region before the medical device is inserted and/or positioned. The insertion means may be a hand, or part thereof. The hand or part thereof may also be used to measure or gauge an inflation pressure. Using the hand to measure an inflation pressure may conveniently comprise using a finger to measure or gauge an inflation pressure.

The body cavity may be a uterus. The medical device may be used to reduce postpartum haemorrhage.

The skilled person will appreciate that all preferred features or embodiments of any aspect of the invention can be applied to all aspects of the invention.

Embodiments of the present invention will now be described, purely by way of example, with reference to the accompanying drawings, in which:

FIG. 1 is a graphic representation of a prior art Bakri Balloon;

FIG. 2 is a diagram showing correctly and incorrectly placed Bakri Balloons according to the prior art;

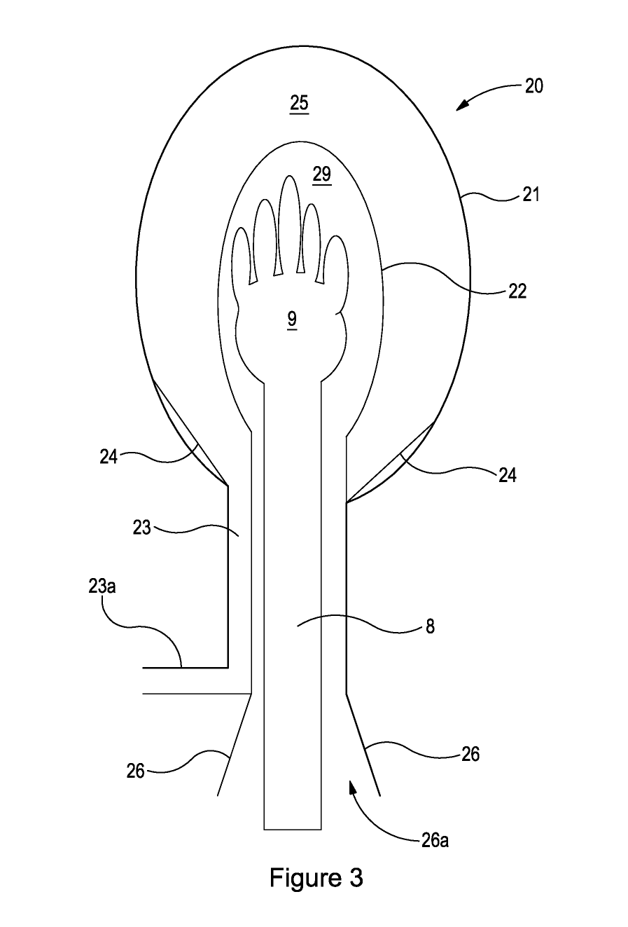

FIG. 3 is a schematic sectional diagram of a medical device for reducing bleeding according to a first embodiment of the invention;

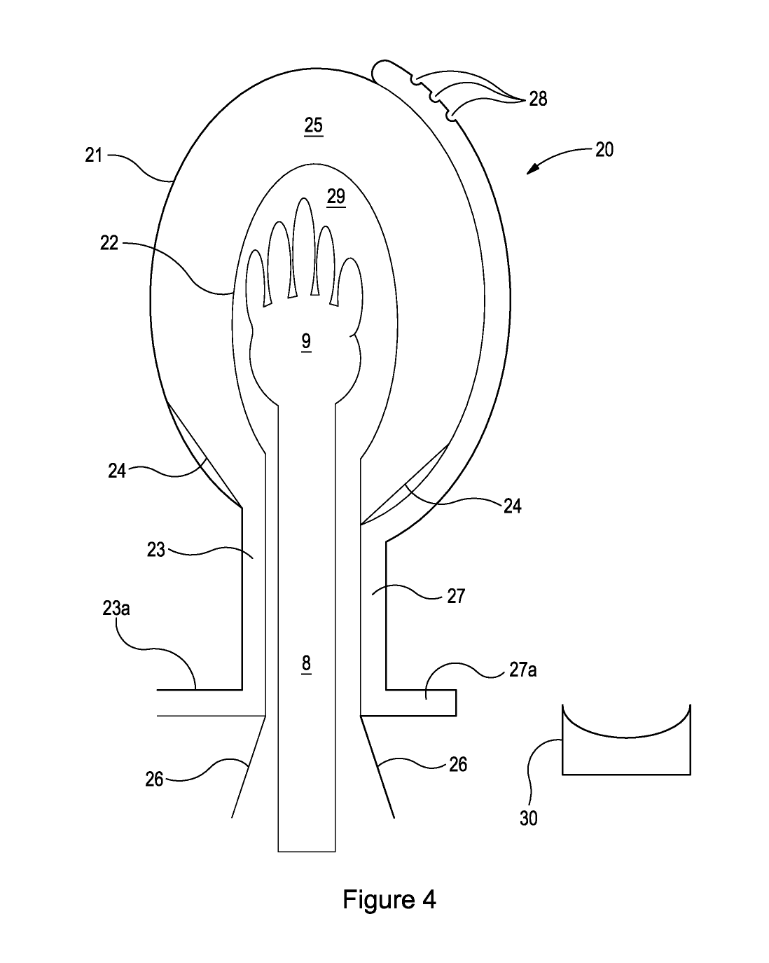

FIG. 4 is a schematic section diagram of a further medical device for reducing bleeding according to a second embodiment of the invention;

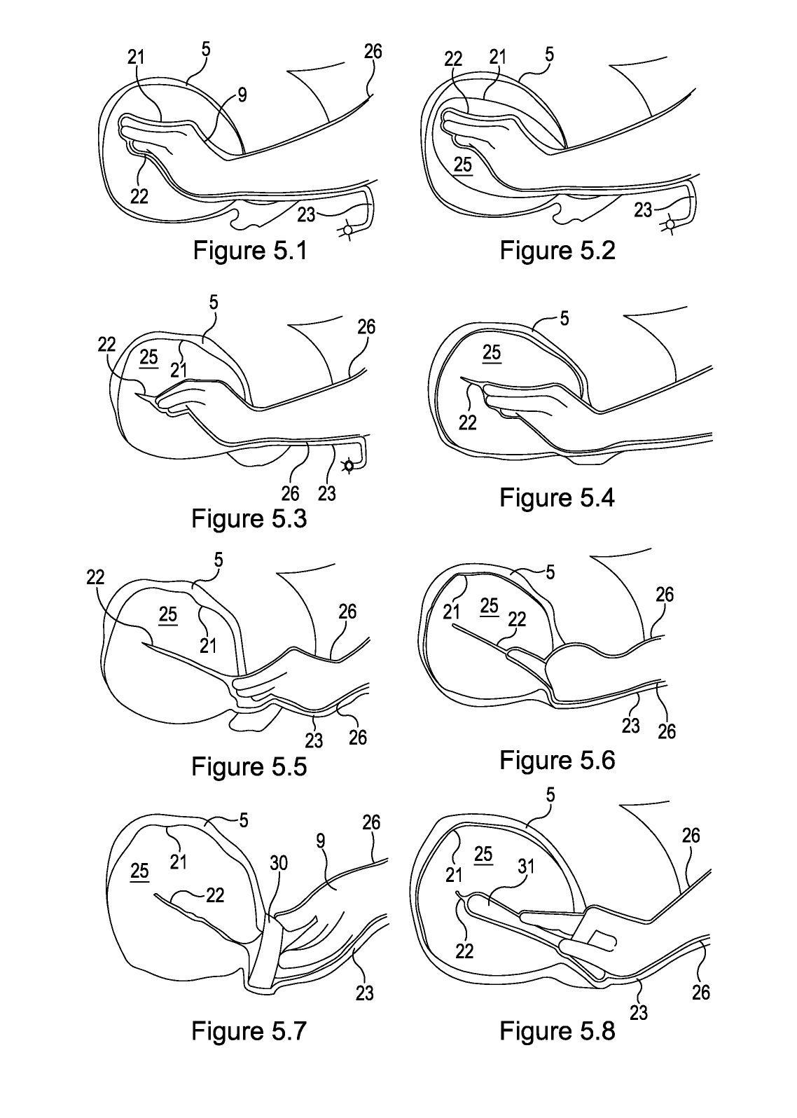

FIGS. 5.1 to 5.8 are a sequence of schematic section drawings showing how a device according to an embodiment may be used;

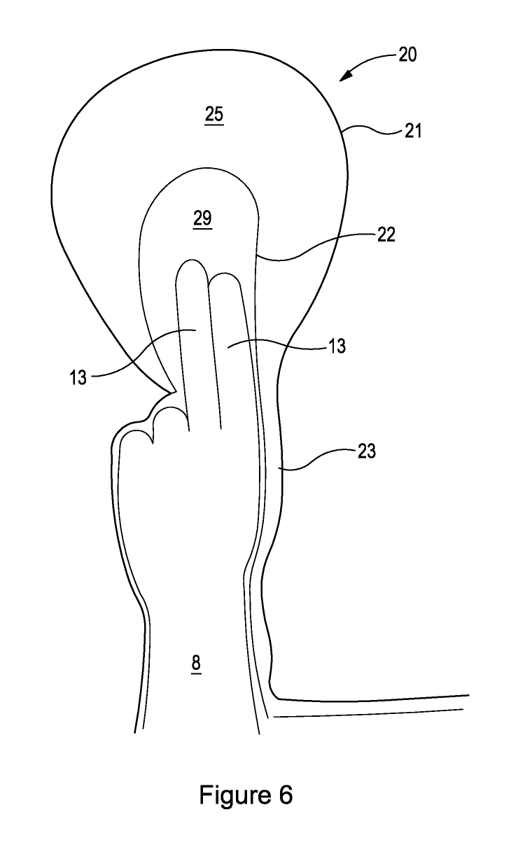

FIG. 6 is a schematic sectional diagram of a medical device for reducing bleeding according to an alternative embodiment of the invention;

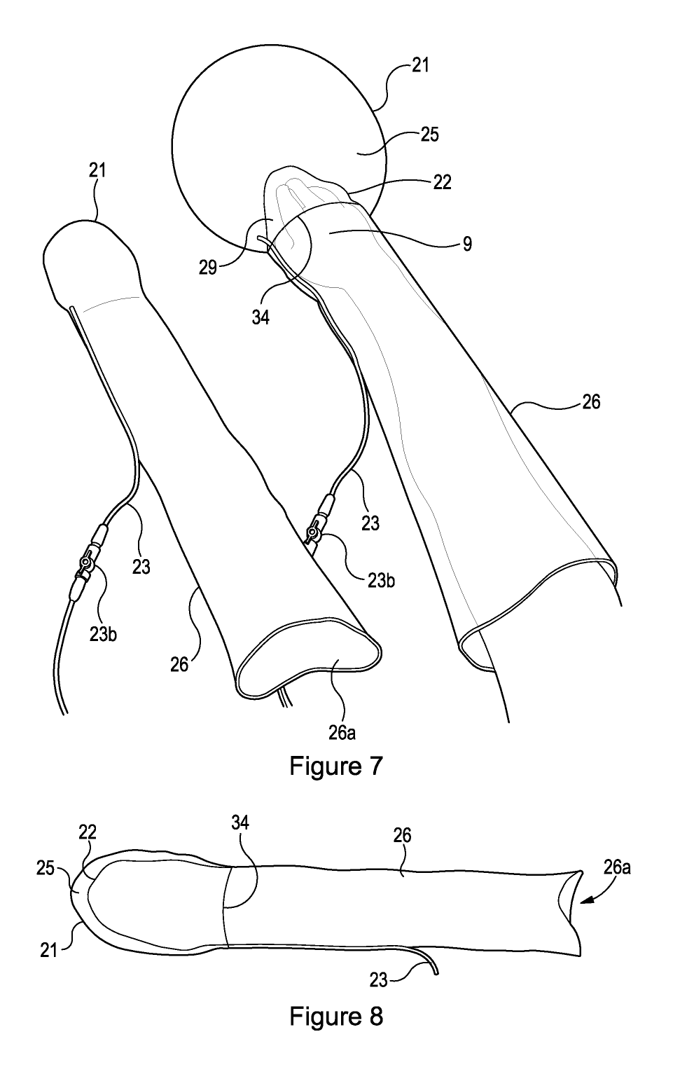

FIG. 7 is a schematic diagram showing two embodiments of the invention, with one embodiment before insertion of an arm or inflation, and the other showing an arm within the device and the inflatable region inflated;

FIG. 8 is a top view of the embodiment shown in FIG. 7.

Referring to FIG. 3, a medical device 20 for reducing bleeding according to an embodiment of the invention is shown, comprising an inner flexible membrane 22, and an outer flexible membrane 21. The inner and outer flexible membranes 22, 21 together define an inflatable region or balloon 25 therebetween, which is inflatable via an inflow channel 23. A further interior region 29 is defined by the inner flexible membrane 22, the interior region 29 being at least partially inside the inflatable region 25. In this embodiment, the device 20 is configured to reduce postpartum haemorrhage, and the inflatable region 25 is designed appropriately so that it will tamponade a womb when inflated.

The interior region 29 communicates to the exterior of the medical device 20, so that guide means may be introduced into the interior region 29 from outside the medical device 20 to enable improved placement thereof.

Conveniently, the interior region 29 communicates to the exterior via a sleeve 26. The sleeve 26 is adjacent to the inflatable region 25 and interior region 29, and communicates from a sleeve opening 26a to the interior region 29. In this embodiment the sleeve 26 is defined by the inner membrane 22, but it will be appreciated that this is not essential, and in other embodiments the sleeve 26 may be at least partly, or entirely defined by the outer membrane 22, or may comprise a separate sleeve flexible membrane.

As shown in FIG. 3, the sleeve 26 and interior region 29 are configured to enable the user or operator of the device to insert their hand 9 into the interior region 29 via the sleeve 26. The medical device 20 of this embodiment is thereby configured with the inner flexible membrane 22 forming a mitten, for receiving a hand of an operator.

The sleeve 26 is conveniently configured to reach up the arm 8 of the operator, approximately as far as the operator's elbow, in use. The patient's body is thereby protected from contact with the operator's body. Furthermore, the exterior of the outer membrane 21 and sleeve 26 may be designed to minimise friction during insertion and positioning of the device.

The inflow channel 23 is conveniently defined, at least in part, by the inner and outer membranes 22, 21. The inflow channel 23 runs adjacent to the sleeve 26 and may be affixed thereto for at least part of its length. This arrangement is preferable, since it results in a smooth exterior profile of the medical device 20, and allows the inflow channel to be formed with minimal cost. In other embodiments the inflow channel 23 may be separate from the sleeve 26 and/or the inner and outer flexible membranes 22, 21. In other embodiments the inflow channel 23 may be separate from the sleeve 26. The inflow channel 23 may for instance be a tube, which may be attached to the outer membrane 21 and/or sleeve 26 along at least part of its length. In some embodiments the inflow channel may be independent of the sleeve 26.

The inflow channel 23 has a fluid input region 23a adjacent to the sleeve opening 26a, formed by a short tube which is separate from the sleeve 26. The fluid input region 23a has an opening through which fluid may be introduced. In some embodiments, a fluid fitting is provided for connecting a syringe for injecting saline solution to inflate the inflatable region 25. In some embodiments a fluid fitting may comprise a valve which can be configured to selectively determine whether fluid can flow into and/or out of the inflow channel.

The outer membrane 21 may comprise a reinforced region 24, which increases the stiffness and/or strength of the outer membrane in a region adjacent to the sleeve 26. This may be advantageous in preventing slippage of the balloon from the womb to the vagina.

FIG. 4 shows an similar medical device 20 according to an embodiment of the invention, further comprising an outflow channel 27, for allowing fluids to drain out through the medical device 20 from the region of the patient's body external to the inflatable region 25. The outflow channel 27 has at least one inflow opening 28 near the top (in use) of the exterior of the inflatable region 25. Conveniently, there are a plurality of inflow openings 28, thereby improving the ease with which fluid may flow into the outflow channel 27.

The outflow channel 27 may conveniently be a tube which is fixed to the outer membrane 21, or may alternatively be integrally formed with the outer membrane 21. In other embodiments, the inflow channel 23 may be a tube that is independent of the outer membrane 21. FIG. 4 shows an arrangement in which the inflow channel 23 and outflow channel 27 are on opposite sides of the sleeve 26, but in other embodiments, the inflow and outflow channel 23, 27 may be adjacent. In some embodiments the outflow channel 27 may be at least partly defined by the inner membrane 22. The outflow channel 27 may be adjacent to, or pass through, the interior region 29.

The outflow channel 27 runs along the sleeve 26, and has a fluid output region 27a adjacent to the sleeve opening 26a. The fluid output region 27a is not fixed to the sleeve 26, and includes an opening from which fluid can exit.

FIG. 4 also shows a cervical sponge plug 30, which may inserted through the sleeve 26 to aid retention of the medical device 20 in the proper position within the body, as shown in FIG. 5.7, and described hereinafter.

FIGS. 5.1 to 5.8 illustrate an example use of a medical device according to an embodiment. It will be appreciated that this example use is merely illustrative, and is not intended to limit the scope of the invention.

FIG. 5.1 shows step 1, in which the operator puts the medical device 20 onto their dominant hand 9, with the hand 9 within the interior region 29. The hand 9, with the medical device 20 thereon is inserted into the uterus 5. The hand 9 is held with the fingertips a few centimetres away from the top of the womb. This step allows the accurate placement of the inflatable region 25 of the medical device.

FIG. 5.2 shows step 2, in which a fluid (for example a saline solution) is gradually introduced into the inflatable region 25, via the inflow channel 23. The hand 9 within the interior region 29 allows the operator to sense the pressure within the inflatable region 25, since this pressure will be acting on their hand through the flexible inner membrane 22. This feature of the medical device allows the pressure exerted by the medical device to be set at an appropriate level, overcoming the limitations of prior art devices. Furthermore, the hand maintains accurate positioning of the medical device 20 during balloon inflation.

FIG. 5.3 shows step 3, in which the balloon has inflated to substantially fill the uterine cavity. The pressure within the inflatable region 25 will subsequently rise. As the filling of the inflatable region 25 nears completion, the hand 9 is slowly withdrawn from the interior region 29, leaving the inflatable region 25 in the proper position within the uterus.

FIG. 5.4 shows step 4, in which the hand 9 is withdrawn past the cervix, into the vagina.

FIG. 5.5 shows step 5, in which the hand 9 in the vagina is used to check the inflatable region 25 of the medical device 20 is still in the correct position within the womb, and that there is no “hour-glass” slippage of the inflatable region 25 into the vagina.

FIG. 5.6 shows step 6, in which the inflation is finalised. As inflation is finalised, the forefinger is inserted and kept within the interior region 29 to provide an assessment of pressure within the womb. The hand 9 is finally removed when the inflatable region is fully inflated and is exerting an appropriate pressure.

FIG. 5.7 shows step 7, in which a cervical sponge plug 30 is inserted through the sleeve 26. The cervical sponge plug helps retain the inflatable region 25 of the medical device 20 in place

FIG. 5.8 shows an optional procedure, in which an insertion means in the form of a elongate device 31 is used to position the inflatable region within the uterus. The elongate device 31 may comprise a plastics material and/or a metal material. It may be appropriate to use an insertion means other than a hand where the region to be treated by tamponade is not accessible by a hand, for example a contracted cervix may prevent the introduction of the operator's hand into the womb. In this case, an insertion means 31 may be held in interior region 29 of the medical device 20 by the operator's hand, and used to accurately position the inflatable region 25. The operator's fore-finger is inserted into the interior region 29 along with the insertion means 31 to provide an assessment of the pressure in the inflatable region 25.

Having inflated the balloon, the inflow channel can be closed to prevent any escape of fluid, thereby ensuring that pressure is maintained. Research has shown that bleeding is extremely unlikely to return after 24 to 48 hours of treatment by tamponade. The inflatable region 25 can therefore be deflated by releasing fluid from the inflow channel after this period has elapsed, and the medical device withdrawn from the womb.

Referring to FIG. 6, an alternative embodiment is shown, in which the interior region 29 is configured to receive at least one finger 13 as the insertion means. Conveniently, two fingers 13 may be used as the insertion means, and the medical device 20 configured accordingly. In some circumstances it may not be possible to insert the whole hand into the uterus, particularly when the womb has already contracted. It will be appreciated that in other embodiments, the medical device may be configured to receive a single finger as the insertion means, or more than two fingers. The use of a device according to the embodiment of FIG. 6 is similar to that described above, with reference to FIGS. 5.1 to 5.8 except that one or more fingers will be inserted into the uterus instead of a whole hand. Accurate placement of the medical device 20 within the uterus can be achieved using the at least one finger 13 within the interior region 29. Likewise, the at least one finger 13 can be used to: maintain the device 20 in position as it is inflated, sense the inflation pressure, and be slowly withdrawn from the uterus as inflation is completed.

Referring to FIGS. 7 and 8, further embodiments of the medical device according to the invention are shown. The device is similar to the embodiment of FIG. 4, and comprises an inner membrane 22, outer membrane 21 and sleeve 26. An inflow channel 23 is provided for inflating the inflatable region 25 defined between the inner and outer membranes 22, 21. The device is placed into the uterus by inserting an arm into the sleeve 26, and is arranged so that the base of the inflatable region 25 is proximal to the knuckles of the arm, in use. The base of the inflatable region 25 is defined by a bond 34 between the inner and outer membranes 22, 21. The bond may be 5-15 cm from the tip of the inner membrane. This is different to the embodiment of FIG. 4, in which the base of the inflatable region is closer to the wrist of the inserted arm.

In the first embodiment the device 20a is shown prior to insertion of the arm. In the second embodiment the device 20b is shown with the arm inserted and the inflatable region 25 inflated.

The device 20a, 20b includes an channel 23 for inflating and deflating the inflatable region 25. A valve 23b is provided in the channel 23 for controlling the flow of fluid through the channel 23.

In some embodiments the medical device may be provided with a further sterile cover, suitable for covering the device as it is inserted into the vagina. The cover may be configured to be ruptured by the insertion device (e.g. a finger) within the vagina, prior to insertion of the inflatable region into the uterus. This may help to keep the device clean and sterile until it reaches the cervix. The sterile cover may comprise a thin polymeric material such as polythene.

A medical device has been disclosed which overcomes a number of limitations with prior art devices. Embodiments of the invention have the potential to significantly improve the quality and reliability of the treatment of postpartum haemorrhage.

In other embodiments, the inflatable region 25 may be designed to tamponade a different part of the body. For example, an embodiment may be designed to tamponade esophageal varices, with an interior region 29 configured to receive an appropriately designed insertion means that enables improved positioning of the device within the esophagus. In other embodiments, the medical device may be designed for use with animals, for example in animals such as dogs or horses.

The skilled person will appreciate that a number of modifications and variations may be made, without departing from the scope of the invention, as defined by the appended claims.

1. A medical device for reducing bleeding comprising:
an outer flexible membrane and an inner flexible membrane, wherein the outer and inner flexible membranes together define an inflatable region between the inner and outer flexible membrane;
an inflow channel for inflation of the inflatable region by communicating a fluid thereto; and
an interior region defined by the inner flexible membrane, the interior region being configured to receive an insertion means for positioning the medical device.

2. The medical device of claim 1, further comprising a sleeve, communicating from a sleeve opening to the interior region.

3. The medical device of claim 1, wherein the interior region is configured to receive at least part of a hand as the insertion means.

4-5. (canceled)

6. The medical device of claim 1, further comprising an outflow channel configured to communicate fluid from an outflow region adjacent to the exterior of the inflatable region to a location remote from the outflow region.

7. The medical device of claim 6, wherein the outflow channel is disposed on the exterior of the inflatable region.

8. The medical device of claim 6, wherein the outflow channel is disposed on the exterior of the sleeve.

9. The medical device of claim 1, wherein the inflow channel is at least partly defined by the inner flexible membrane and/or the outer flexible membrane.

10. The medical device of claim 1, further comprising a reinforced region of the outer flexible membrane.

11. The medical device of claim 10, wherein the reinforced region defines at least part of the inflatable region adjacent to the sleeve.

12. The medical device of claim 1, wherein the sleeve is at least partly defined by the inner flexible membrane and/or the outer flexible membrane.

13. (canceled)

14. The medical device of claim 1, further comprising a sterile cover for protecting the outer membrane from contamination as it is positioned.

15. (canceled)

16. The medical device of claim 1, wherein a base of the inflatable region is 5-15 cm from a tip of the inner flexible membrane.

17. (canceled)

18. A medical apparatus comprising a medical device according to claim 1 and: a sponge or an inflatable plug configured to be inserted into the medical device to aid retention of the inflatable region, and/or an insertion means configured to enable insertion and/or positioning of the inflatable region within a region of the body into which a hand cannot be placed.

19-21. (canceled)

22. A method of reducing bleeding, comprising the steps of: inserting and/or positioning a medical device according to claim 1 within a body cavity, wherein inserting comprises using an insertion means received within the interior region of the medical device, and inflating the medical device.

23. The method of claim 22, further comprising the steps of deflating the medical device and removing it from the body cavity.

24. The method of claim 22, wherein the insertion means is received within the interior region before the medical device is inserted and/or positioned.

25. The method of claim 22, wherein the insertion means comprises at least part of a hand.

26. (canceled)

27. The method of claim 25, comprising using at least part of the hand to measure an inflation pressure.

28. The method of claim 22, wherein the body cavity is a uterus.

29. The method of claim 28, wherein the medical device is used to reduce postpartum haemorrhage.

30. (canceled)
